(19) United States
(12) Patent Application Publication
(10) Pub. No.: US 2026/0053495 A1
(43) Pub. Date: Feb. 26, 2026

(54) MINIMALLY INVASIVE SURGICAL METHOD FOR HAGLUND'S DEFORMITY AND PARATENON PRESERVATION

(71) Applicant: William H. Simon, Virginia Beach, VA (US)

(72) Inventor: William H. Simon, Virginia Beach, VA (US)

(21) Appl. No.: 19/378,090

(22) Filed: Nov. 3, 2025

Related U.S. Application Data
(63) Continuation-in-part of application No. 18/340,376, filed on Jun. 23, 2023, now Pat. No. 12,458,342.
Application No. 18/340,376 is a division of application No. 18/162,332, filed on Jan. 31, 2023, now Pat. No. 11,723,651.
(60) Provisional application No. 63/267,364, filed on Jan. 31, 2022.

(51) Int. Cl. A61B 17/04

(52) U.S. Cl. CPC A61B 17/0469; A61B 17/0401; A61B 2017/0496

(57) ABSTRACT

The present disclosure provides a surgical method for treating Haglund's deformity and posterior insertional calcific Achilles pathology. The method comprises positioning a patient prone under general anesthesia, using fluoroscopic guidance to identify and mark anatomical landmarks on a calcaneus, and creating two proximal surgical incisions. The method includes separating medial and lateral edges of an Achilles tendon from overlying paratenon using a paratenon fascial elevator to preserve paratenon vascularity. The method comprises placing loop locking sutures along medial and lateral tendon edges using an Achilles suture passer device and creating a mini-open lateral incision for direct access to the Haglund's deformity. The method includes resecting a bony deformity and debriding calcific deposits under direct visualization, securing the Achilles tendon using knotless suture anchors with a rip-stop loop locking technique, and creating subcutaneous tunnels for crossed suture tape placement that provides compression fixation.

### CROSS REFERENCE TO RELATED APPLICATIONS

This application is a continuation-in-part of and claims priority to U.S. application No. Ser. No. 18/340,376, titled Surgical Devices And Methods For Achilles Tendon Repair, filed Jun. 23, 2023, and granted as U.S. Pat. No. 12,458,342 on Nov. 4, 2025, which is a divisional of U.S. application No. Ser. No. 18/162,332, filed Jan. 31, 2023, and granted as U.S. Pat. No. 11,723,651 on Aug. 15, 2023, which is a nonprovisional filing of and claims priority to U.S. Provisional Application No. 63/267,364, filed Jan. 31, 2022, all entitled Surgical Devices And Methods For Achilles Tendon Repair. The above provided applications are hereby incorporated by reference in their entirety.

### FIELD OF INVENTION

The present disclosure relates to surgical techniques for treating foot and ankle pathology, and more particularly to a minimally invasive surgical method for treating Haglund's deformity and posterior insertional calcific Achilles pathology that preserves paratenon integrity while providing secure tendon fixation through advanced suture constructs.

### BACKGROUND

Haglund's deformity, also known as “pump bump,” is a bony enlargement on the posterior aspect of the calcaneus that can cause pain and functional impairment in patients. This condition frequently occurs in conjunction with posterior insertional calcific Achilles pathology, creating a complex clinical scenario that presents challenges for surgical management.

Traditional surgical approaches for treating Haglund's deformity and associated Achilles pathology typically involve extensive open posterior incisions with complete detachment of the Achilles tendon from its calcaneal insertion. These conventional techniques often require aggressive soft tissue dissection that can compromise surrounding anatomical structures, leading to complications including wound healing problems, nerve injury, adhesion formation, and increased risk of tendon rupture.

The paratenon serves as a structure that provides vascular supply to the Achilles tendon through a network of longitudinally oriented blood vessels. Traditional surgical approaches often compromise this structure through aggressive dissection or complete division, potentially leading to ischemia, adhesion formation, and impaired healing. The extensive dissection required in conventional approaches can damage a substantial portion of the paratenon's vascular network, which contributes to compromised tendon healing.

Additionally, conventional fixation methods using traditional sutures that may experience failure in diseased or compromised tendon tissue, potentially resulting in repair failure and the need for revision surgery. While traditional open approaches provide adequate visualization of the pathology, they can result in soft tissue trauma, prolonged healing times, and suboptimal functional outcomes due to the disruption of anatomical structures.

There exists a need in the field for surgical techniques that can preserve paratenon vascularity while providing enhanced fixation strength, reduce soft tissue trauma through less invasive approaches, and optimize healing potential through advanced suture constructs. Such techniques would address the limitations of current surgical methods while maintaining the ability to adequately treat the underlying pathology.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a surgical method for treating Haglund's deformity and posterior insertional calcific Achilles pathology is provided. The surgical method comprises positioning a patient prone under general anesthesia with or without use of a radiolucent operating table. The method includes using fluoroscopic guidance to identify and mark anatomical landmarks on a calcaneus. The method comprises creating two proximal surgical incisions that are in an approximate range of 1 to 3 centimeters each. The method includes separating medial and lateral edges of an Achilles tendon from overlying paratenon using a paratenon fascial elevator in order to preserve paratenon vascularity. This is the same fascial elevator described in both U.S. Pat. Nos. 12,458,342 and 11,723,651. The method comprises placing loop locking sutures along medial and lateral tendon edges using an Achilles suture passer device. The method includes creating a mini-open lateral incision for direct access to the Haglund's deformity. The method comprises resecting a bony deformity and debriding calcific deposits under direct visualization. The method includes securing the Achilles tendon using knotless suture anchors with a rip-stop loop locking technique. The method comprises creating subcutaneous tunnels for crossed suture tape placement that provides compression fixation.

According to other aspects of the present disclosure, the surgical method may include one or more of the following features. The surgical method may preserve paratenon in order to maintain up to 90% of original vascular supply to the Achilles tendon. The loop locking sutures may be constructed using high-strength fiber tape with ultimate tensile strength exceeding 200 pounds. The high-strength fiber tape may include attached shuttle sutures for loop locking suture construction. Placing the loop locking sutures may capture both gastrocnemius fascia and soleus fascia of a triceps surae tendon complex. The mini-open lateral incision may be in a range of approximately 3-5 centimeters in length. The rip-stop loop locking technique may distribute load across multiple tissue planes to prevent suture failure in compromised tendon tissue. The crossed suture tape placement may provide load distribution, compression, redundancy, and stability to a repair. The knotless suture anchors may be constructed from bioabsorbable materials selected from PLLA or PEEK. The paratenon fascial elevator may feature a blunt, curved tip designed to create a surgical working plane while maintaining paratenon vascular network integrity.

According to another aspect of the present disclosure, a surgical system for treating Haglund's deformity and posterior insertional calcific Achilles pathology is provided. The surgical system comprises a paratenon fascial elevator having a blunt, curved tip for separating tendon edges from paratenon while preserving vascularity. The system includes an Achilles suture passer device for precise placement of loop locking sutures. This is the same Achilles suture passer as described in both U.S. Pat. Nod. 12,458,342 and 11,723,651. The system comprises high-strength fiber tape with ultimate tensile strength exceeding 200 pounds. The system includes knotless suture anchors constructed from bioabsorbable materials. The system comprises curved hemostats for creating subcutaneous tunnels.

According to other aspects of the present disclosure, the surgical system may include one or more of the following features. The Achilles suture passer device may feature a curved needle design that follows a natural contour of the tendon. The high-strength fiber tape may include attached shuttle sutures for loop locking suture construction. The attached shuttle sutures may facilitate passage of fiber tape through tendon tissue and enable creation of a loop locking configuration. The knotless suture anchors may provide secure bone fixation without bulk associated with traditional knotted repairs. The bioabsorbable materials may be selected from one or more polymers including PLLA and/or PEEK.

According to another aspect of the present disclosure, a method for preserving paratenon vascularity during Achilles tendon surgery is provided. The method comprises creating proximal surgical incisions positioned to accommodate specialized instruments. The method includes inserting a paratenon fascial elevator through proximal incisions. The method comprises advancing the paratenon fascial elevator distally to create a smooth dissection plane between tendon substance and paratenon. The method includes maintaining said paratenon as an intact, vascularized envelope around an Achilles tendon. The method comprises preserving a paratenon's dual function as both a vascular conduit and gliding surface.

According to other aspects of the present disclosure, the method for preserving paratenon vascularity may include one or more of the following features. A paratenon preservation technique may maintain up to 90 percent of an original vascular supply compared to traditional approaches that preserve only 40-50 percent. The paratenon fascial elevator may create a dissection plane without disrupting vascular connections within the paratenon. The paratenon fascial elevator may feature a blunt, curved tip designed to separate tissue planes through gentle dissection rather than cutting.

More specifically, the present disclosure provides for a surgical method for treating Haglund's deformity and posterior insertional calcific Achilles pathology, comprising:
positioning a patient prone under general anesthesia;
using fluoroscopic guidance to identify and mark anatomical landmarks on a calcaneus;
creating two proximal surgical incisions;
separating medial and lateral edges of an Achilles tendon from overlying paratenon using a paratenon fascial elevator to preserve paratenon vascularity;
placing loop locking sutures along medial and lateral tendon edges using an Achilles suture passer device;
creating a mini-open lateral incision for direct access to the Haglund's deformity;
resecting a bony deformity and debriding calcific deposits under direct visualization;
securing the Achilles tendon using knotless suture anchors with a rip-stop loop locking technique; and
creating subcutaneous tunnels for crossed suture tape placement that provides compression fixation.

The two proximal surgical incisions are in an approximate range of 1 to 3 centimeters each.

In addition, the surgical method preserves paratenon to maintain up to 90% of original vascular supply to the Achilles tendon.

Here, the loop locking sutures are constructed using high-strength fiber tape with ultimate tensile strength exceeding 200 pounds.

The high-strength fiber tape includes attached shuttle sutures for loop locking suture construction.

Placing the loop locking sutures correctly captures both gastrocnemius fascia and soleus fascia of a triceps surae tendon complex.

Here the mini-open lateral incision is in a range of approximately 3-5 centimeters in length.

In addition, the rip-stop loop locking technique distributes load across multiple tissue planes to prevent suture failure in compromised tendon tissue.

Importantly, the crossed suture tape placement provides load distribution, compression, redundancy, and stability to a repair.

The knotless suture anchors are constructed from bioabsorbable materials selected from PLLA or PEEK or other suture materials.

In another embodiment the present disclosure includes a surgical system for treating Haglund's deformity and posterior insertional calcific Achilles pathology, comprising:
a paratenon fascial elevator having a blunt, curved tip for separating tendon edges from paratenon while preserving vascularity;
an Achilles suture passer device for precise placement of loop locking sutures;
high-strength fiber tape with ultimate tensile strength exceeding 200 pounds;
knotless suture anchors constructed from bioabsorbable materials; and
curved hemostats for creating subcutaneous tunnels.

The use of an Achilles suture passer device features a curved needle design that follows a natural contour of the tendon.

Here, high-strength fiber tape includes attached shuttle sutures for loop locking suture construction.

The use of an attached shuttle sutures facilitate passage of fiber tape through tendon tissue and enable creation of a loop locking configuration.

Here the knotless suture anchors provide secure bone fixation without bulk associated with traditional knotted repairs.

For this disclosure, the bioabsorbable materials are selected from one or more polymers including PLLA and PEEK and/or other suture materials.

In an additional embodiment, a method for preserving paratenon vascularity during Achilles tendon surgery is described as;
creating proximal surgical incisions positioned to accommodate specialized instruments;
inserting a paratenon fascial elevator through the proximal incisions;
advancing the paratenon fascial elevator distally to create a smooth dissection plane between tendon substance and paratenon;
maintaining the paratenon as an intact, vascularized envelope around an Achilles tendon; and
preserving a paratenon's dual function as both a vascular conduit and gliding surface.

This paratenon preservation technique maintains up to 90 percent of an original vascular supply compared to traditional approaches that preserve only 40-50 percent.

The paratenon fascial elevator creates the dissection plane without disrupting vascular connections within the paratenon.

In addition, the paratenon fascial elevator features a blunt, curved tip designed to separate tissue planes through gentle dissection rather than cutting.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a posterior view of an ankle region showing surgical markings for Haglund's deformity treatment, according to aspects of the present disclosure.

FIG. 2 is an x-ray radiographic image a lateral view of the posterior ankle region showing Haglund's deformity and associated pathology including the “pump bump” and a bone spur inside the tendo according to an embodiment.

FIG. 3 illustrates a schematic of an x-ray radiographic image lateral view of the posterior ankle region that illustrates the proposed bone resection of Haglund's deformity and traction spurring.

FIG. 4 illustrates a posterior view of the ankle region showing loop locking sutures positioned along tendon edges, according to aspects of the present disclosure.

FIG. 5 illustrates a posterior view of the ankle region showing a surgical approach using loop locking stitches for treating Haglund's deformity, according to an embodiment.

FIG. 6 illustrates a top view of the Achilles tendon during a final tensioning stage prior to final fixation, according to an embodiment.

FIG. 7 illustrates a lateral view of the foot and ankle following completion of the surgical procedure with 3 loop locking stitches and a fourth loop locking stitch that resides over a proximal suture anchor. according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

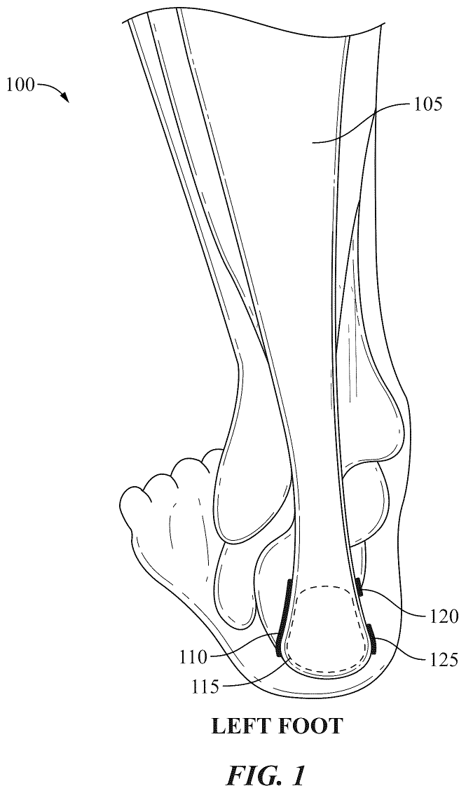

Referring to FIG. 1, a posterior view of the ankle region [100] shows an Achilles tendon [105] and calcaneus with surgical markings that are shown as dotted lines along the posterior surface of the calcaneus [115] that guide the minimally invasive surgical approach. The patient may be positioned prone under general anesthesia, and in some cases, a radiolucent operating table may be used to facilitate fluoroscopic guidance during the procedure. The radiolucent operating table may provide enhanced visualization capabilities, though the surgical method may be performed without such specialized positioning equipment.

As shown in FIG. 1, two incision sites are marked on either side of the Achilles tendon in the upper portion of the ankle region one is the medial proximal incision marked site [120] which is roughly 2 cm in length and the other is the medial distal incision marked site [125] that is roughly 1 cm in length. These proximal surgical incisions may range from approximately 1 to 3 centimeters each as required per patient, providing flexibility in the surgical approach based on patient anatomy and surgeon preference. The incision positioning may accommodate specialized instruments for paratenon preservation and suture placement while maintaining access to the surgical working space.

With continued reference to FIG. 1, dotted lines indicate planned resection markings on the posterior aspect of the calcaneus [115], corresponding to the location of the Haglund's deformity. The planned resection lines may delineate the area of bony prominence to be addressed during the surgical procedure. Fluoroscopic guidance determine proper resection angle using Fowler's angle and calcaneal pitch angle parameters, which provide anatomical reference points for achieving appropriate bone correction while preserving structural integrity of the calcaneus. In addition to the medial proximal and distal marked sites [120, 125] there is also shown a lateral skin incision marking and eventual incision [110] show. It should be emphasized this is for a left foot surgical repair as shown here in FIG. 1.

The anatomical representation in FIG. 1 shows the natural contour of the Achilles tendon as the tendon tapers toward the calcaneal insertion. The Achilles tendon extends downward from the posterior aspect of the leg to the insertion point on the posterior calcaneus, demonstrating the anatomical relationship between the tendon and underlying bone structure. The tapering configuration influences suture placement and fixation techniques used during the surgical procedure.

As further shown in FIG. 1, the surgical markings provides guidance for the minimally invasive approach, with the proximal incisions positioned to preserve paratenon integrity while enabling access for specialized surgical instruments. The mini-open lateral skin incision [110] normally ranges from approximately 3-5 centimeters in length, providing direct access to the Haglund's deformity for resection and debridement procedures. The variable incision sizing allows surgeons to adapt the approach based on individual patient anatomy and the extent of pathological tissue requiring treatment.

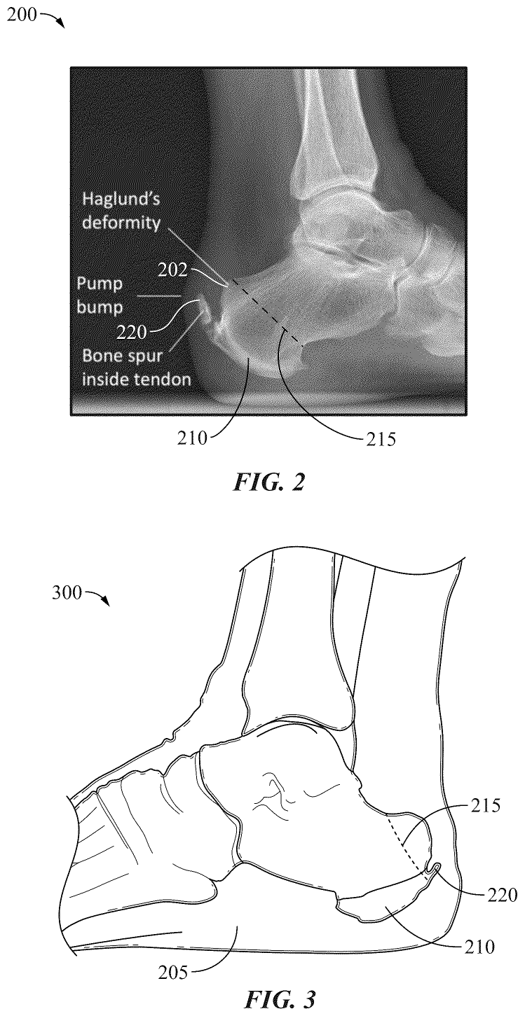

Next, referring to FIG. 2, an x-ray radiographic image lateral view of the posterior ankle region demonstrates the pathological features characteristic of Haglund's deformity and associated conditions. The figure displays the bony prominence that appears as an enlarged posterior-superior aspect of the calcaneus [202], which creates mechanical irritation and functional impairment in affected patients. The bony enlargement extends beyond the normal anatomical contours of the calcaneus, creating a prominence that interferes with normal tendon function and causes pain during activities.

As shown in FIG. 2, the soft tissue prominence labeled as “pump bump” [210] represents the external manifestation of the condition that patients experience as a visible and palpable enlargement at the posterior heel region. The pump bump [210] results from the underlying bony deformity and associated soft tissue inflammation, creating a compound pathological condition that affects both skeletal and soft tissue structures. The external prominence causes difficulties with footwear fitting and contributes to ongoing irritation and discomfort.

With continued reference to FIG. 2, calcific deposits are indicated as “bone spur inside tendon” demonstrating the insertional calcific pathology that frequently accompanies Haglund's deformity. These calcific deposits develops within the Achilles tendon insertion area, creating additional mechanical and biological challenges for surgical treatment. The calcific pathology compromises tendon tissue quality and affects the holding strength of suture constructs, which influences surgical planning and fixation techniques.

The insertional calcific pathology shown in FIG. 2 represents a degenerative process that affects the tendon-bone interface, potentially leading to weakened tissue that are more susceptible to suture failure during repair procedures. The calcific deposits create areas of compromised tissue quality that may require specialized fixation techniques to achieve secure tendon reattachment. The presence of calcific pathology indicate the need for thorough debridement during surgical treatment to remove diseased tissue and create a healthy environment for healing.

As further shown in FIG. 2, the lower portion provides radiographic visualization of the bony structures and demonstrates the relationship between the calcaneus and the overlying soft tissues. Also shown is the reaction line of bone deformity [215] as well as areas where intratendon calcifications occur. The radiographic view reveals the extent of bony deformity and assists in surgical planning by showing the three-dimensional characteristics of the pathological condition. The imaging provides information about bone density, cortical integrity, and the spatial relationship between the bony prominence and surrounding anatomical structures.

The schematic of the radiographic image shown in FIG. 3 again shows the Haglund's deformity that enables assessment of the calcaneal bone quality and guides anchor placement during surgical fixation procedures. Here is shown the proposed bone lateral incision placement [215] resection of the Haglund's deformity and where the traction spurring is located. The imaging show areas of sclerotic bone that provide enhanced holding strength for suture anchors, as well as areas of compromised bone that correspond to the bone spur [210] inside the Achilles tendon that require modified fixation techniques. The relationship between the calcaneus and overlying soft tissues are evaluated to determine the extent of soft tissue involvement and the degree of mechanical impingement caused by the bony deformity and especially the shown pump bump [220].

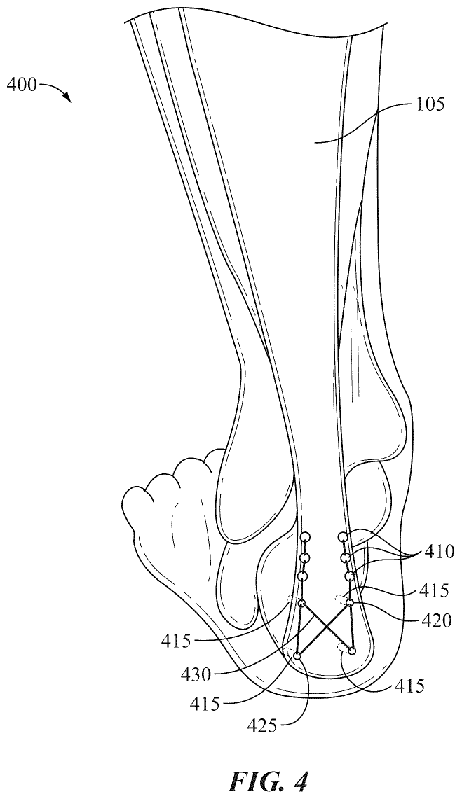

Referring to FIG. 4, a posterior view of the ankle region [400] illustrates the placement of three proximal loop locking sutures [410] along both the medial and lateral edges of the Achilles tendon. The sutures are positioned to extend longitudinally from proximal to distal regions of the tendon, creating a bilateral suture configuration that engages the tendon tissue along its length. The longitudinal placement provides distributed fixation points that enhance the mechanical stability of the repair construct.

As shown in FIG. 4, the loop locking sutures [410] follow the natural contour of the Achilles tendon as the tendon transitions from its broader proximal region to its narrower distal insertion area. The suture placement conforms to the anatomical shape of the tendon, which allows for optimal tissue engagement while maintaining the structural integrity of the tendon fibers. The contour-following placement distributes mechanical forces along the natural load-bearing pathways of the tendon structure.

With continued reference to FIG. 4, the suture configuration demonstrate multiple points of tissue engagement distributed along the tendon edges. These multiple engagement points create a construct that distributes mechanical loads across multiple tissue planes rather than concentrating stress at individual locations. The distributed loading pattern reduces the likelihood of suture failure at any single point and provides enhanced holding strength in compromised or diseased tendon tissue.

The loop locking sutures shown in FIG. 4 are placed using an Achilles suture passer device that may feature a curved needle design. The curved needle design follows the natural contour of the tendon during suture placement, which facilitates accurate positioning of the sutures while minimizing tissue trauma. The curved configuration allows the needle to navigate the anatomical contours of the tendon and surrounding structures, providing precise suture placement in the desired tissue planes.

As further shown in FIG. 4, the high-strength fiber tape [430] used for the loop locking sutures have two free ends of the tape that are brought back through the Achilles tendon and a loop locking stitch at the location of the proximal knotless sutures [415] acting as suture anchors. Here are shown 4 such anchors; all distal to the proximal three loop locking stitches [410]. The procedure to provide this technique requires using attached using attached shuttle sutures that facilitate loop locking suture construction. It should be noted that the distal region here represents diseased tendon area which when anchored as shown will utilize the healthier Achilles tendon region above it to provide much greater strength and support for both tensioning and final surgical completion. The shuttle sutures enable passage of the fiber tape through tendon tissue and assist in creating the loop locking configuration. The attached shuttle sutures provide a mechanism for threading the high-strength fiber tape through the tissue while maintaining proper suture orientation and tension during placement. Here there are three loop locking stitches in healthy tendon above the zone of the diseased or otherwise damaged tendon. The two medial incisions [420] which are roughly 2 cm are shown along with the four suture anchors [415] (two proximal and two distal) that have a double row crossing compression construct so that the last loop locked stitch-a fourth stitch as shown here-) is located over the proximal suture anchors (often referred to as Kustler's Anchors). Here the two free ends of the suture tape [430] coming out of the suture anchors are then brought back through the underlying Achilles tendon [105] and the last loop locked stitches are connected to the two knotless distal and two proximal suture anchors that creates a rip-stop construct where the last loop lock stitch is a portion of the rip-stop that preserves the reconnection tensioning and repair of the Achilles tendon with the calcaneus.

The loop locking suture construction demonstrated in FIG. 4 captures both gastrocnemius fascia and soleus fascia of the triceps surae tendon complex. The multi-layer capture technique may engage multiple fascial planes within the tendon structure, which provides enhanced fixation strength compared to single-layer suture placement. The engagement of both fascial layers distribute loads across the different components of the tendon complex, greatly improving the biomechanical properties of the repair without involving any portion of the paratenon. This technique allows complete blood flow to be maintained throughout the paratenon to ensure extremely (compared with conventional techniques) rapid healing.

The suture arrangement illustrated in FIG. 4 [425] represents the positioning of the repair construct prior to final fixation to the calcaneal bone surface. The bilateral suture placement create a framework for tendon reattachment that maintains the anatomical relationship between the tendon and the underlying bone structure. The loop locking configuration also shown here as provides a secure attachment mechanism that resists the tensile forces encountered during normal ankle motion and rehabilitation activities.

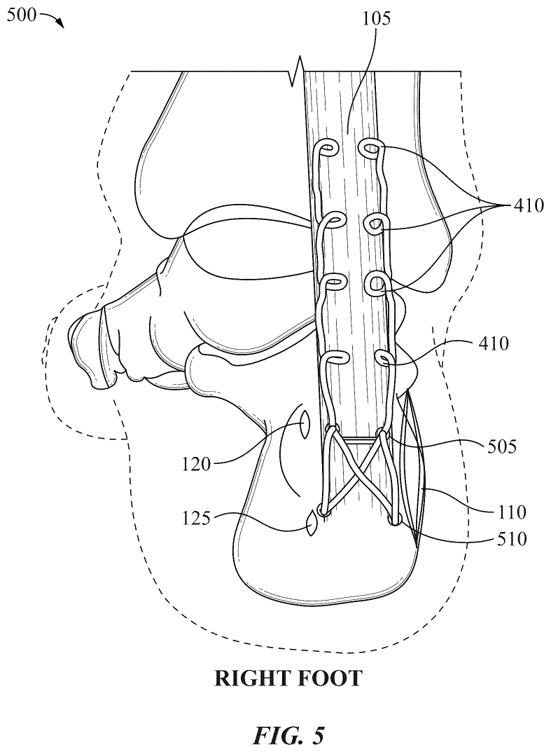

Referring to FIG. 5, a posterior view of the ankle region of a right foot and [500] demonstrates the surgical approach that combines direct access to the Haglund's deformity [202] with paratenon preservation techniques. The lateral longitudinal incision [110] provides direct access to the bony deformity while maintaining the integrity of surrounding vascular structures. The incision placement allows surgeons to visualize and address the pathological tissue while preserving anatomical structures that are compromised by more extensive surgical approaches.

As shown in FIG. 5, two smaller medial incisions [120, 125] accommodate specialized surgical instruments including a paratenon fascial elevator and suture placement devices. These medial incisions are positioned to provide access for instrument insertion while minimizing disruption to the paratenon envelope that surrounds the Achilles tendon. The incision positioning creates working space for the specialized instruments while maintaining the vascular network that supplies the tendon tissue.

With continued reference to FIG. 5, high-strength fiber tape as loop locking sutures [410] (here four are shown) are positioned along the medial and lateral edges of the healthy Achilles tendon in a loop locking configuration. The suture loop locking positioning captures the tendon tissue in preparation for reattachment to the prepared bone surface following completion of the bony resection and debridement procedures. The four (4) shown loop locking sutures [410] are provided “blindly” whereas the 5th proximal and 6th distal loop lock [505, 510] respectively is shown at the site of the future to be inserted knotless suture anchor.

The loop locking configuration illustrated in FIG. 5 captures both gastrocnemius fascia and soleus fascia of the triceps surae tendon complex. The multi-fascial layer engagement provides enhanced fixation strength by distributing loads across different components of the tendon structure. The suture placement engages these fascial layers while maintaining the anatomical organization of the tendon complex, which preserves the biomechanical properties of the repaired structure.

As further shown in FIG. 5, the minimally invasive technique preserves the paratenon while providing access for suture placement through the strategically positioned incisions. The paratenon remains as an intact, vascularized envelope around the Achilles tendon during the surgical procedure. The preservation of this structure maintains both the vascular supply and the gliding properties that are compromised by more extensive surgical dissection techniques.

The surgical approach demonstrated in FIG. 5 may utilize a paratenon fascial elevator that creates a dissection plane without disrupting vascular connections within the paratenon. The paratenon fascial elevator separates the tendon edges from the overlying paratenon through controlled dissection that maintains the integrity of the vascular network. The dissection technique preserves the longitudinally oriented blood vessels that provide nutrition to the tendon tissue throughout the healing process.

With continued reference to FIG. 5, the paratenon fascial elevator that is utilized features a blunt, curved tip designed to separate tissue planes through gentle dissection rather than cutting. The blunt tip configuration allows the instrument to create working space between anatomical structures without causing tissue damage that compromises vascular supply. The curved design follows the natural contours of the tendon and paratenon interface, facilitating smooth separation of tissue planes while maintaining structural integrity.

The gentle dissection technique enabled by the specialized elevator design preserves the delicate vascular connections that supply the Achilles tendon. The blunt tip creates separation through mechanical displacement rather than tissue division, which maintain the continuity of blood vessels within the paratenon structure. The preservation of these vascular connections support tendon healing and reduce the risk of ischemic complications that occur with more aggressive dissection techniques.

Here, the paratenon preservation technique maintain up to 90 percent of the original vascular supply compared to traditional approaches that preserve only 40-50 percent. The enhanced vascular preservation results from the specialized dissection technique that maintains the integrity of the paratenon envelope and its associated blood vessel network. The improved vascular preservation provide enhanced healing potential and reduce complications associated with compromised tendon blood supply.

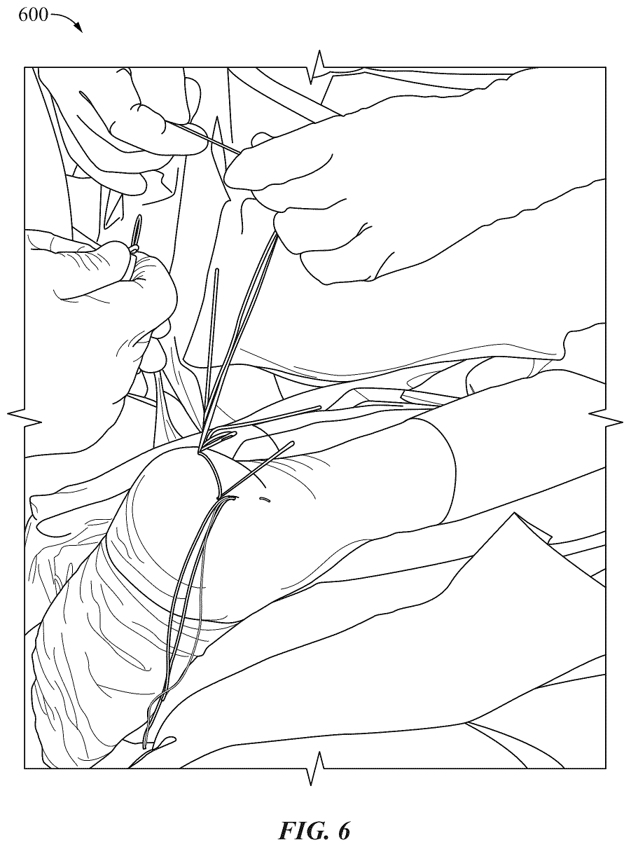

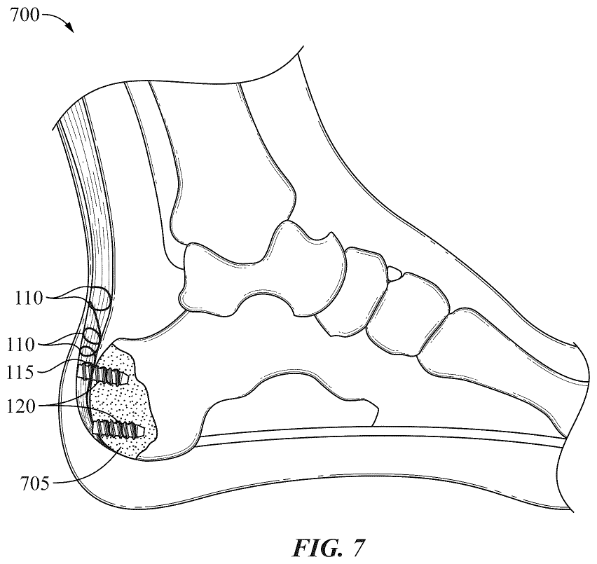

The 90 percent vascular preservation achieved through the technique illustrated in both FIG. 5 and FIG. 6 represents a substantial improvement over conventional surgical approaches. Traditional methods involve extensive dissection that disrupts a significant portion of the paratenon's vascular network, potentially leading to ischemia and impaired healing. The minimally invasive approach maintains the majority of the original blood supply, which supports biological healing processes and improves functional outcomes following surgical treatment.

As further shown by FIG. 5, the tendon width is demonstrated through the positioning of the suture constructs relative to the medial and lateral borders of the tendon substance. The suture placement is positioned to engage the tendon tissue near the borders while maintaining adequate distance from the edges to prevent tissue failure during loading. The positioning relative to the tendon borders optimizes the mechanical properties of the repair while preserving the anatomical structure of the tendon.

The facilitated passage enabled by the shuttle sutures may allow for precise placement of the high-strength fiber tape within the tendon tissue. The shuttle sutures provides control over the threading process, which ensures that the fiber tape is positioned in the desired tissue planes and orientations. The controlled placement optimizes the mechanical properties of the repair by ensuring that the high-strength fiber tape engages the tendon tissue in locations that provides maximum holding strength and load distribution.

As further shown in FIG. 5, the loop locking configuration created through the shuttle-facilitated passage provides a secure attachment mechanism that resists the forces encountered during tendon healing and rehabilitation. The loop structure creates multiple points of contact between the fiber tape and the tendon tissue, which distributes mechanical loads across a broader area of tissue engagement. The distributed loading pattern reduces the likelihood of suture failure and provides enhanced security for the tendon repair construct.

Referring now to FIG. 6, a top view of the Achilles tendon during the final tensioning stage demonstrates the posterior aspect of the calcaneus with the Achilles tendon positioned above the bone surface. The view shows the spatial relationship between the tendon structure and the underlying calcaneal bone following completion of the bony resection and debridement procedures. The positioning reflects the anatomical alignment that is achieved after removal of the pathological tissue and preparation of the bone surface for tendon reattachment.

As shown in FIG. 6, multiple suture constructs extend along the medial and lateral edges of the Achilles tendon in their tensioned state. The suture constructs appear as loop locking configurations that have been placed through the tendon tissue and positioned for final fixation to the prepared bone surface. The tensioned state demonstrates the mechanical loading of the suture constructs applied to achieve secure tendon-bone contact and compression at the repair interface with proper tensioning and no concern with regard to further damaging already diseased tissue.

With continued reference to FIG. 6, the loop locking configurations are positioned to provide secure fixation of the tendon to the bone surface before being locked into position with fixation anchors. The positioning demonstrates the final arrangement of the suture constructs prior to anchor placement, showing how the sutures engage both the tendon tissue and the prepared bone surface. The secure fixation positioning creates the mechanical foundation for the repair construct that may maintain tendon-bone contact throughout the healing process.

The tensioned suture constructs illustrated in FIG. 6 demonstrates the application of controlled mechanical forces that compress the tendon against the prepared calcaneal surface. The tensioning creates intimate contact between the tendon tissue and the bone surface, which facilitate biological integration during the healing process. The controlled application of tension ensures adequate compression while avoiding excessive forces that compromise tissue viability or cause mechanical failure of the suture constructs.

As further shown in FIG. 6, the arrangement illustrates the positioning of the repair construct immediately before final fixation is achieved through anchor placement. The pre-fixation positioning allows for final adjustments to suture tension and alignment before the anchors are deployed to lock the construct in place. The positioning represents the culmination of the suture placement and tensioning procedures, creating the final configuration that is secured through the anchor fixation system.

The final tensioning stage demonstrated in FIG. 6 utilizes knotless suture anchors that are constructed from bioabsorbable materials. The bioabsorbable materials are selected from PLLA (poly L-lactic acid) and/or PEEK (polyetherether ketone) or other suitable suture materials, which may provide different mechanical and biological properties for the fixation system. The material selection is based on factors including desired resorption characteristics, mechanical strength requirements, and compatibility with the healing environment.

PLLA anchors may provide bioabsorbable fixation that gradually transfer loads to the healing tendon-bone interface as the anchor material is resorbed by biological processes. The PLLA material offer controlled degradation characteristics that may allow for initial mechanical fixation followed by gradual load transfer to the biological repair. The resorption process may occur over a timeframe that may coincide with the biological healing and integration of the tendon-bone interface.

PEEK anchors provide biocompatible fixation with enhanced mechanical properties that are suitable for applications requiring sustained mechanical support. The PEEK material offers superior strength characteristics compared to traditional bioabsorbable materials, which may be beneficial in cases involving compromised bone quality or high mechanical demands. The biocompatible properties of PEEK allow for long-term implantation without adverse tissue reactions while maintaining mechanical integrity throughout the healing process. Other suitable suture materials (conventional nylons and others) are also possible for use.

The knotless design of the suture anchors shown in the final tensioning stage of FIG. 7 provide secure bone fixation without the bulk associated with traditional knotted repairs. The knotless mechanism may eliminate the need for manual knot tying, which reduce surgical time and provide more consistent tension maintenance compared to hand-tied knots. The reduced bulk may minimize potential irritation of surrounding soft tissues and allow for improved tissue healing around the fixation points.

As further shown in FIG. 7, the knotless anchor system is shown in conjunction with a lateral view of the foot after resection of the Haglund's Deformity and debridement of the Achilles Tendon [700]. This system works in conjunction with the tensioned suture constructs to provide secure fixation while maintaining the compression forces applied during the tensioning stage. The suture anchors [115, 120] deployment locks the suture constructs in their tensioned position, preserving the mechanical loading that creates tendon-bone compression. The locking mechanism prevents relaxation of the suture tension that otherwise occur with traditional fixation methods, maintaining consistent compression forces throughout the healing period.

Referring again to FIG. 7, the lateral view of the foot and ankle [700] demonstrates the completed surgical repair following treatment of Haglund's deformity and associated Achilles pathology. The calcaneus shows the contours that result from surgical resection of the bony deformity, with the posterior aspect of the bone modified to eliminate the pathological prominence that characterized the original condition. The bone surface appears smooth and contoured and illustrates the position of the suture anchors within the diseased portion of the Achilles tendon and the repaired portion of the calcaneus [705] that provides an appropriate foundation for tendon reattachment while eliminating the mechanical irritation that contributed to the patient's symptoms.

As shown in FIG. 7, the Achilles tendon [115] extends from the posterior aspect of the leg down to the insertion point on the posterior calcaneus, demonstrating the restored anatomical relationship between the tendon and the underlying bone structure [705]. The tendon positioning reflects the successful reattachment achieved through the surgical procedure, with the tendon secured against the prepared bone surface in a configuration that restores normal biomechanical function. The anatomical alignment demonstrates the correction of any displacement or malalignment that have resulted from the original pathological condition.

With continued reference to FIG. 7, loop locking stitches [110] (in this case 3 of them) are visible along the Achilles tendon, demonstrating the suture pattern used to secure the tendon to the calcaneal insertion site. The loop locking configuration show the distributed suture placement that engages the tendon tissue at multiple points along its length and width. The suture pattern reflects the bilateral placement technique that captures both medial and lateral aspects of the tendon, creating a secure fixation construct that resist the various forces encountered during normal ankle motion.

The loop locking stitches [110] illustrated in FIG. 7 demonstrate the final configuration of the high-strength fiber tape sutures following tensioning and fixation procedures. The sutures that exist are shown from a cross-sectional view and appear integrated with the tendon tissue, showing how the loop locking technique engage multiple tissue planes to distribute mechanical loads across the repair construct. The suture integration reflects the successful capture of both gastrocnemius and soleus fascial layers, creating a multi-layer fixation optimally utilizing the healthy Achilles tendon [105] that enhances the holding strength of the repair.

As further shown in FIG. 7, two fixation suture anchors [115, 120] (there are at least 4 total-only 2 are shown from the side view here) penetrate through the calcaneus, representing the anchor points for the suture construct that secure the loop locking stitches to the bone. The suture anchors are positioned to provide optimal mechanical advantage for the suture fixation while maintaining adequate bone purchase for secure anchoring. The suture anchor placement reflects the strategic positioning that distribute fixation loads across structurally sound areas of the calcaneus while avoiding interference with surrounding anatomical structures.

The fixation suture anchors [115, 120] shown in FIG. 7 represents knotless suture anchors that provide secure bone fixation without the bulk associated with traditional knotted repairs. The anchors are deployed through the calcaneal bone to engage the cortical and cancellous bone structures, creating secure fixation points for the suture constructs. The anchor positioning demonstrates the biomechanically advantageous placement that optimizes the load transfer between the suture constructs and the underlying bone structure.

With continued reference to FIG. 7, the final configuration of the repair demonstrates the integration of the tendon reattachment with the calcaneus following resection of the bony deformity. The repair configuration shows how the surgical procedure addresses both the bony pathology through resection and the soft tissue pathology through secure reattachment. The integrated repair, that greatly improves upon conventional techniques, completely restores the anatomical relationship between the tendon and bone while eliminating the mechanical factors that contributed to the original pathological condition.

The tendon reattachment illustrated in FIG. 7 demonstrates the restoration of the normal insertion anatomy following removal of calcific deposits and pathological tissue. The reattachment show the tendon secured against healthy bone tissue that provides a suitable environment for biological integration and healing. The attachment configuration reflects the successful debridement of compromised tissue and the creation of a repair environment that both strengthens the surgically repaired reattachment and enhances the support of long-term healing and functional restoration.

As further shown in FIG. 7, the relationship between the tendon, fixation hardware, and underlying bone structure demonstrates the biomechanical integration achieved through the surgical procedure. The hardware positioning shows how the fixation anchors engage the bone structure to secure the suture constructs that attaches to the tendon. The integrated system creates a mechanically stable construct that maintains tendon-bone contact while allowing for the biological processes that lead to permanent integration of the repair.

The completed repair [700] demonstrated in FIG. 7 shows the lateral perspective of the relationship between the various components of the fixation system. The lateral view provides visualization of how the loop locking sutures, fixation anchors, and bone preparation work together to create a comprehensive repair construct. The perspective demonstrates the spatial arrangement of the repair components and shows how the fixation system maintains the anatomical alignment of the tendon-bone interface.

With continued reference to FIG. 7, the final configuration represents the culmination of the minimally invasive surgical technique that completely preserves paratenon vascularity without any possible damage to the paratenon as the paratenon is not ever touched while achieving secure tendon fixation. The completed repair demonstrates how the various surgical components work together to address the pathological condition while maintaining the biological environment that supports healing. The configuration shows the integration of the paratenon preservation technique with the advanced fixation methods to create a repair that optimizes both mechanical stability, full restored contour removing the Haglund's deformity while allowing for fully restored blood flow and biological healing potential to the damaged and normally diseased Achilles tendon tissue after reattachment to the calcaneus.

The completed surgical repair [700] illustrated in FIG. 7 demonstrates the successful treatment of both the bony deformity and the associated soft tissue pathology through a coordinated surgical approach. The repair shows how the resection of the Haglund's deformity eliminates the mechanical irritation while the secure tendon reattachment restores normal biomechanical function. The integrated treatment addresses the complex pathophysiology of the condition while preserving anatomical structures that are compromised by more extensive surgical approaches.

A number of implementations have been described. Nevertheless, it will be understood that various modifications may be made without departing from the spirit and scope of the disclosure. Accordingly, other implementations are within the scope of the following claims.

1. A surgical method for treating Haglund's deformity and posterior insertional calcific Achilles pathology, comprising:
positioning a patient prone under general anesthesia;
using fluoroscopic guidance to identify and mark anatomical landmarks on a calcaneus;
creating two proximal surgical incisions;
separating medial and lateral edges of an Achilles tendon from overlying paratenon using a paratenon fascial elevator to preserve paratenon vascularity;
placing loop locking sutures along medial and lateral tendon edges using an Achilles suture passer device;
creating a mini-open lateral incision for direct access to said Haglund's deformity;
resecting a bony deformity and debriding calcific deposits under direct visualization;
securing said Achilles tendon using knotless suture anchors with a rip-stop loop locking technique; and
creating subcutaneous tunnels for crossed suture tape placement that provides compression fixation.

2. The surgical method of claim 1, wherein said two proximal surgical incisions are in an approximate range of 1 to 3 centimeters each.

3. The surgical method of claim 1, wherein said surgical method preserves paratenon to maintain up to 90% of original vascular supply to said Achilles tendon.

4. The surgical method of claim 1, wherein said loop locking sutures are constructed using high-strength fiber tape with ultimate tensile strength exceeding 200 pounds.

5. The surgical method of claim 4, wherein said high-strength fiber tape includes attached shuttle sutures for loop locking suture construction.

6. The surgical method of claim 1, wherein placing said loop locking sutures captures both gastrocnemius fascia and soleus fascia of a triceps surae tendon complex.

7. The surgical method of claim 1, wherein said mini-open lateral incision is in a range of approximately 3-5 centimeters in length.

8. The surgical method of claim 1, wherein said rip-stop loop locking technique distributes load across multiple tissue planes to prevent suture failure in compromised tendon tissue.

9. The surgical method of claim 1, wherein said crossed suture tape placement provides load distribution, compression, redundancy, and stability to a repair.

10. The surgical method of claim 1, wherein said knotless suture anchors are constructed from bioabsorbable materials selected from PLLA and/or PEEK and/or other suture materials.

11. A surgical system for treating Haglund's deformity and posterior insertional calcific Achilles pathology, comprising:
a paratenon fascial elevator having a blunt, curved tip for separating tendon edges from paratenon while preserving vascularity and creates a surgical working plane for an Achilles suture passer device for precise placement of loop locking sutures;
high-strength fiber tape with ultimate tensile strength exceeding 200 pounds;
knotless suture anchors constructed from bioabsorbable materials; and
curved hemostats for creating subcutaneous tunnels.

12. The surgical system of claim 11, wherein said Achilles suture passer device features a curved needle design that follows a natural contour of the tendon.

13. The surgical system of claim 11, wherein said high-strength fiber tape includes attached shuttle sutures for loop locking suture construction.

14. The surgical system of claim 13, wherein said attached shuttle sutures facilitate passage of fiber tape through tendon tissue and enable creation of a loop locking configuration.

15. The surgical system of claim 11, wherein said knotless suture anchors provide secure bone fixation without bulk associated with traditional knotted repairs.

16. The surgical system of claim 11, wherein said bioabsorbable materials are selected from one or more polymers including PLLA and/or PEEK and/or other suture materials.

17. A method for preserving paratenon vascularity during Achilles tendon surgery, comprising:
creating proximal surgical incisions positioned to accommodate specialized instruments;
inserting a paratenon fascial elevator through said proximal incisions;
advancing said paratenon fascial elevator proximally to create a smooth dissection plane between tendon substance and paratenon;
maintaining said paratenon as an intact, vascularized envelope around an Achilles tendon; and
preserving a paratenon's dual function as both a vascular conduit and gliding surface.

18. The method of claim 17, wherein said paratenon preservation technique maintains up to 90 percent of an original vascular supply compared to traditional open surgical approaches that preserve only 40-50 percent.

19. The method of claim 17, wherein said paratenon fascial elevator creates said dissection plane without disrupting vascular connections within said paratenon.

20. The method of claim 19, wherein said paratenon fascial elevator features a blunt, curved tip designed to separate tissue planes through gentle dissection rather than cutting.
